(19) United States
(12) Patent Application Publication
(10) Pub. No.: US 2016/0051606 A1
(43) Pub. Date: Feb. 25, 2016

(54) COMPOSITION FOR LIFE EXTENSION CONTAINING GINSENG FRUIT EXTRACT

(71) Applicant: AMOREPACIFIC CORPORATION, Seoul (KR)

(72) Inventors: Si Young CHO, Yongin-si, Gyeonggi-do (KR); Jue Won KIM, Yongin-si, Gyeonggi-do (KR); Chan Woong PARK, Yongin-si, Gyeonggi-do (KR); Dae Bang SEO, Yongin-si, Gyeonggi-do (KR); Eun Gyung CHO, Yongin-si, Gyeonggi-do (KR); Wan Gi KIM, Yongin-si, Gyeonggi-do (KR); Sang Jun LEE, Yongin-si, Gyeonggi-do (KR); Hyun Hee KIM, Yongin-si, Gyeonggi-do (KR)

(73) Assignee: Amorepacific Corporation, Seoul (KR)

(21) Appl. No.: 14/781,160

(22) PCT Filed: Apr. 3, 2014
(86) PCT No.: PCT/KR2014/002886

§ 371 Date: Nov. 5, 2015

(30) Foreign Application Priority Data
Apr. 3, 2013 (KR) 10-2013-0036393
Apr. 1, 2014 (KR) 10-2014-0038804

(51) Int. Cl. A61K 36/258; A23L 1/28; A23L 2/52; A61K 31/704

(52) U.S. Cl. CPC A61K 36/258; A61K 31/704; A23L 1/28; A23L 2/52; A23V 2002/00

(57) ABSTRACT

The present invention relates to a composition for life extension and particularly to a composition for life extension containing a ginseng fruit extract. The present invention can extend the life of an individual by containing the ginseng fruit extract as an active ingredient and thus is medically highly useful.

### TECHNICAL FIELD

The present disclosure relates to a composition for life extension, specifically to a composition for life extension, which contains an extract of a ginseng fruit.

### BACKGROUND ART

In general, aging is known to be affected by various factors including genetics, environments and lifestyles. Among them, the genetic factors are known to affect only about 30%. Accordingly, depending on how the acquired aging factors are controlled, control of aging, e.g., prevention or delaying of aging could be possible, and as a result, lifespan could be extended. Since the extension of lifespan can lead to decrease in diseases as confirmed by the compression of morbidity hypothesis, successful aging or healthy aging with high physical, cognitive and social functioning and free from disabilities can be possible.

### DISCLOSURE

### Technical Problem

The present disclosure is directed to providing a composition for extending the lifespan of a subject.

### Technical Solution

In an aspect, the present disclosure provides a composition for life extension, which contains a ginseng fruit extract as an active ingredient.

### Advantageous Effects

The present disclosure provides a composition containing a ginseng fruit extract as an active ingredient, which can extend the lifespan of a subject and thus is highly useful medicinally. Specifically, the ginseng fruit extract as an active ingredient of the composition of the present disclosure has been confirmed to be able to lower mortality and extend lifespan in fruit fly and Caenorhabditis elegans, which are highly genetically homologous to human. In addition, since the ginseng fruit extract as the active ingredient of the composition of the present disclosure has been found to be able to reduce the number of specific cells known as aging markers (Mechanisms of Ageing and Development (2004), 125; 381-390) in mouse, the composition of the present disclosure can extend the lifespan of mammals.

### BRIEF DESCRIPTION OF DRAWINGS

FIG. 1 shows a survival curve of fruit flies fed with a ginseng fruit extract.

FIG. 2 shows the median lifespan of nematodes fed with a ginseng fruit extract.

### BEST MODE

In the present disclosure, “life extension” means extension of the lifespan of a subject as compared to a natural state. For example, the “life extension” may mean the extension of the lifespan of a subject which is determined by congenital factors such as genetic factors or acquired factors. Specifically, the “life extension” may include extension of the lifespan of a subject through adequate control of the factors that regulate lifespan associated with the genetic factors of the subject, e.g., the expression level of related genes or the production level of related proteins, or extension of the lifespan of a subject through adequate control of the factors that regulate lifespan associated with the acquired factors such as environment. For example, the extension of lifespan may mean delaying the natural death of an organism.

In the present disclosure, the “ginseng” may be so-called Korean ginseng, or Panax ginseng C. A. Meyer.

In the present disclosure, the “ginseng fruit” includes both a live ginseng fruit and a dried ginseng fruit extract. Because the period during which the “ginseng fruit” can be harvested is very limited, a harvested ginseng fruit is dried to improve its storage property. It is obvious to those skilled in the art that the live ginseng fruit and the dried ginseng fruit extract have the same composition.

In the present disclosure, the “ginseng fruit extract” may include any substance obtained by treating a ginseng fruit with an extraction method known in the art, without limitation.

Specifically, the “extract” may include any extract obtained from a ginseng fruit regardless of the extraction method, extraction solvent, extracted ingredients or the type of the extract. In addition, the “extract” includes a broad concept, including any substance that can be obtained by processing or otherwise treating a primarily obtained ginseng fruit extract otherwise. More specifically, the processing or treatment may be fermenting or enzymatically treating the ginseng fruit extract, although not being limited thereto.

Specifically, in the present disclosure, the “ginseng fruit extract” may be (a) a live ginseng fruit or a primary extract obtained by adding a solvent to a dried ginseng fruit extract.

In the present disclosure, the ginseng fruit may be one containing only the pulp and rind of a ginseng fruit with the seeds having been removed.

In the present disclosure, the dried ginseng fruit extract may be any one obtained by drying a ginseng fruit according to a method known in the art without limitation. Specifically, the drying may be sunlight drying, hot air drying, evaporation drying, spray drying or freeze-drying, although not being limited thereto.

In the present disclosure, the solvent may be one or more selected from a group consisting of water, a C₁-C₆ lower alcohol, acetone, ether, ethyl acetate, diethyl ether, methyl ethyl ketone and chloroform. Specifically, the solvent may be ethanol, although not being limited thereto.

The primary extract may also include (a′) a secondary extract obtained by adding the solvent to a live ginseng fruit or a dried ginseng fruit extract, and filtering and concentrating the same under reduced pressure. The concentration under reduced pressure may be performed by any method known in the art without limitation.

For example, the “ginseng fruit extract” may be a tertiary extract obtained by removing oil-soluble components from the primary extract or the secondary extract. The oil-soluble components may be removed by any method known in the art without limitation. Specifically, ether may be treated, although not being limited thereto.

In the composition according to an aspect of the present disclosure, the “ginseng fruit extract” may be a quaternary extract obtained by extracting the tertiary extract with a C₁-C₅ lower alcohol. The alcohol may include methanol, ethanol or butanol.

The ginseng fruit extract is different from ginseng root extract in ingredients and composition. For example, it may have a ratio of a protopanaxadiol (PD)-based ginsenoside to a protopanaxatriol (PT)-based ginsenoside of 0.5-2.5. This ratio has never been found in the ginseng root extract. That is to say, the ginseng fruit has a very high ginsenoside content than the ginseng root and is entirely different from the ginseng root in the composition of protopanaxatriol (PT)-based ginsenosides Rb1, Rb2, Rc and Rd and protopanaxadiol (PD)-based ginsenosides Re, Rg1 and Rg2. In this aspect, the ratio of a protopanaxadiol (PD)-based ginsenoside to a protopanaxatriol (PT)-based ginsenoside may be 0.5 or greater, 0.55 or greater, 0.6 or greater, 0.65 or greater, 0.69 or greater, 0.7 or greater or 0.73 or greater, and 2.5 or smaller, 2.3 or smaller, 2.0 or smaller, 1.95 or smaller, 1.9 or smaller, 1.85 or smaller, 1.8 or smaller, 1.75 or smaller, 1.73 or smaller, 1.7 or smaller, 1.69 or smaller, 1.65 or smaller, 1.6 or smaller, 1.55 or smaller, 1.5 or smaller, 1.45 or smaller, 1.4 or smaller, 1.35 or smaller, 1.3 or smaller, 1.25 or smaller, 1.2 or smaller, 1.15 or smaller, 1.1 or smaller, 1.05 or smaller, 1.0 or smaller, 0.95 or smaller, 0.9 or smaller or 0.85 or smaller.

In addition, the ginseng fruit extract may be rich in one or more mineral selected from a group consisting of potassium, calcium, iron, phosphorus, magnesium and zinc and/or one or more vitamin selected from a group consisting of vitamin A, vitamin B₁, vitamin B₂, vitamin B₆, vitamin C, vitamin E, vitamin K, vitamin B₃ (niacin), vitamin B₅ (pantothenic acid) and vitamin B₉ (folic acid). The abundant mineral and vitamin components are also the characteristics of ginseng fruit distinguished from ginseng root.

Accordingly, there is a significant difference in the previously known composition, which contains a ginseng root extract as an active ingredient, and the composition according to the present disclosure, which contains a ginseng fruit extract as an active ingredient.

In an aspect, the present disclosure relates to a composition for life extension, which contains a ginseng fruit extract.

The composition according to an aspect of the present disclosure may contain 0.001-80 wt % of the ginseng fruit extract based on the total weight of the composition. This range is not only adequate to achieve the effect intended in the present disclosure but also satisfies both the stability and safety of the composition. The range may also be appropriate in terms of cost effectiveness. Specifically, when the ginseng fruit extract is less than 1 wt %, a sufficient lifespan extending effect cannot be achieved. And, when it exceeds 80 wt %, safety and stability of the composition may be unsatisfactory. In this aspect, the composition according to an aspect of the present disclosure may contain 5-60 wt % or 10-30 wt % of the ginseng fruit extract based on the total weight of the composition.

The composition according to an aspect of the present disclosure may have a ratio of a protopanaxadiol (PD)-based ginsenoside to a protopanaxatriol (PT)-based ginsenoside of 0.5-2.5. This ratio has never been found in the ginseng root extract. That is to say, the ginseng fruit has a very high ginsenoside content than the ginseng root and is entirely different from the ginseng root in the composition of protopanaxatriol (PT)-based ginsenosides Rb1, Rb2, Rc and Rd and protopanaxadiol (PD)-based ginsenosides Re, Rg1 and Rg2. In this aspect, the ratio of a protopanaxadiol (PD)-based ginsenoside to a protopanaxatriol (PT)-based ginsenoside may be 0.5 or greater, 0.55 or greater, 0.6 or greater, 0.65 or greater, 0.69 or greater, 0.7 or greater or 0.73 or greater, and 2.5 or smaller, 2.3 or smaller, 2.0 or smaller, 1.95 or smaller, 1.9 or smaller, 1.85 or smaller, 1.8 or smaller, 1.75 or smaller, 1.73 or smaller, 1.7 or smaller, 1.69 or smaller, 1.65 or smaller, 1.6 or smaller, 1.55 or smaller, 1.5 or smaller, 1.45 or smaller, 1.4 or smaller, 1.35 or smaller, 1.3 or smaller, 1.25 or smaller, 1.2 or smaller, 1.15 or smaller, 1.1 or smaller, 1.05 or smaller, 1.0 or smaller, 0.95 or smaller, 0.9 or smaller or 0.85 or smaller.

The composition according to an aspect of the present disclosure may be a food composition. In another aspect of the present disclosure, the food composition may be an indulgence food or health food composition.

The formulation of the food composition is not particularly limited. For example, it may be formulated into a tablet, a granule, a powder, a liquid such as a drink, a caramel, a gel, a bar, etc. For each formulation of the food composition, those skilled in the art may adequately select and add the ingredients commonly used in the art in addition to the active ingredient considering the particular formulation or purpose of use. A synergic effect may be achieved when different ingredients are used together.

Determination of the administration dosage of the active ingredient is within the level of those skilled in the art. A daily administration dosage may be, for example, 0.1-5000 μg/kg/day, more specifically 50-500 μg/kg/day, although not being limited thereto. The administration dosage may vary depending on various factors, including the age and health condition of the subject, the presence of complication(s), etc.

The composition according to an aspect of the present disclosure may be a pharmaceutical composition.

The pharmaceutical composition according to an aspect of the present disclosure may be administered orally or parenterally, e.g., rectally, topically, transdermally, intravenously, intramuscularly, intraperitoneally or subcutaneously.

Formulations for oral administration may include a tablet, pill, a soft or hard capsule, a granule, a powder, a dust, a liquid, an emulsion or a pellet, although not being limited thereto. These formulations may contain, in addition to the active ingredient, a diluent (e.g., lactose, dextrose, sucrose, mannitol, sorbitol, cellulose or glycine), a lubricant (e.g., silica, talc, stearic acid or polyethylene glycol) or a binder (e.g., magnesium aluminum silicate, starch paste, gelatin, tragacanth, methyl cellulose, sodium carboxymethyl cellulose or polyvinylpyrrolidine). As occasion demands, they may contain a pharmaceutical additive such as a disintegrant, an absorbent, a colorant, a flavor, a sweetener, etc. The tablet may be prepared by a commonly used mixing, granulating or coating method.

Formulations for parenteral administration may include an eye drop, an injection, a medicinal drop, a lotion, an ointment, a gel, a cream, a suspension, an emulsion, a suppository, a patch or a spray, although not being limited thereto.

The administration dosage of the active ingredient of the pharmaceutical composition according to an aspect of the present disclosure may vary depending on the age, sex and body weight of the subject, the particular pathological condition and severity thereof, administration route or the discretion of a diagnoser. Determination of the administration dosage based on these factors is within the level of those skilled in the art. A daily administration dosage may be, for example, 0.1-100 μg/kg/day, more specifically 5-50 μg/kg/day, although not being limited thereto.

In another aspect, the present disclosure may relate to a use of a ginseng fruit extract in preparation of a composition for life extension. The use includes both therapeutic and non-therapeutic uses.

In another aspect, the present disclosure may relate to a method for administering a ginseng fruit extract to a subject for life extension.

### MODE FOR INVENTION

Hereinafter, the constitution and effect of the present disclosure will be described through examples and test examples. However, the following examples and test examples are for illustrative purposes only and it will be apparent to those of ordinary skill in the art that the scope of the present disclosure is not limited by them.

### EXAMPLE 1

### Preparation of Ginseng Fruit Extract

1) Preparation of ginseng fruit: The fruit of live ginseng (Panax ginseng C. A. Meyer) was harvested. After separating and removing seeds, a crude ginseng fruit was prepared by sunlight drying or hot air drying the pulp and rind of the ginseng fruit.

2) Preparation of ginseng fruit extract: 3 L of ethanol was added to 1 kg of the crude ginseng fruit. After reflux extracting at 70° C. for 4 hours and then filtering, 300 g of a ginseng fruit extract was obtained by concentrating under reduced pressure at 45° C.

### EXAMPLE 2

### Preparation of Live Ginseng Fruit Extract

The fruit of live ginseng (Panax ginseng C. A. Meyer) was harvested. After separating and removing seeds, a crude ginseng fruit was prepared by mixing the juice, pulp and rind of the ginseng fruit. 3 L of ethanol was added to 1 kg of the crude ginseng fruit. After reflux extracting at 70° C. for 4 hours and then filtering, 30 g of a live ginseng fruit extract was obtained by concentrating under reduced pressure at 45° C.

### COMPARATIVE EXAMPLE 1

### Preparation of Ginseng Root Extract

A ginseng root was hot air dried. After adding 3 L of water to 1 kg of the drying product, followed by reflux extraction and filtration, a 300 g of a ginseng root extract was obtained by concentrating under reduced pressure.

### TEST EXAMPLE 1

### Comparison of Components of Ginseng Fruit and Ginseng Root Extracts

1. Analysis of Ginsenoside (Ginseng Saponin) Components of Ginseng Fruit and Ginseng Root

Each of the extracts obtained in Examples 1-2 and Comparative Example 1 were treated with ether to remove oil-soluble components. After extracting a crude saponin with ethanol and concentrating, the ginsenoside components were analyzed by HPLC. The result is shown in Table 1.

TABLE 1

| | Example 1 | Example 2 | Comparative Example 1 |
|---|---|---|---|
| Total saponin content | 33.42% | 31.08 | 16.70% |
| (dry weight) | | | |
| PD/PT ratio | 0.73 | 0.69 | 3.23 |

It was confirmed that the ginseng fruit extract prepared in Example 1 has a total saponin (total ginsenoside) content corresponding to 2 times or greater as compared to that of the ginseng root extract prepared in Comparative Example 1.

In addition, the ginseng fruit extract prepared in Example 1 and the live ginseng fruit extract prepared in Example 2 show little difference in total saponin contents. Accordingly, it will be obvious to those skilled in the art that the ginseng fruit extract according to the present disclosure also includes the live ginseng fruit extract.

It was also confirmed from Table 1 that the ginseng fruit extract prepared in Example 1 and the ginseng root extract prepared in Comparative Example 1 show distinct difference in the ratio of the PD (protopanaxadiol)-based ginsenosides Rb1, Rb2, Rc and Rd to the PT (protopanaxatriol)-based ginsenosides Re, Rg1 and Rg2 (PD/PT ratio) and that the ginseng fruit extracts prepared in Example 1 and the live ginseng fruit extract prepared in Example 2 show little difference in total saponin contents. Accordingly, it will be obvious to those skilled in the art that the ginseng fruit extract according to the present disclosure also includes the live ginseng fruit extract.

### TEST EXAMPLE 2

### Analysis of Mineral Components of Ginseng Fruit Extract

To confirm whether the dried ginseng fruit extract and the live ginseng fruit extract prepared in Example 1 and Example 2 have the same composition, the vitamin and mineral components were analyzed. The result is shown in Table 2.

TABLE 2

| Components | Example 1 | Example 2 | Components | Example 1 | Example 2 |
|---|---|---|---|---|---|
| Potassium | 5865.57 | 4977.50 | Magnesium | 354.38 | 297.54 |
| (mg/100 g) | | | (mg/100 g) | | |
| Calcium | 819.26 | 914.22 | Zinc (mg/100 g) | 178.49 | 201.25 |
| (mg/100 g) | | | | | |
| Iron (mg/100 g) | 59.31 | 52.11 | Vit. A (μg/100 g, | 213.11 | 145.85 |
| | | | RE) | | |
| Phosphorus | 187.17 | 156.95 | Vit. B₁ (mg/100 g) | 12.29 | 10.09 |
| (mg/100 g) | | | | | |
| Vit. B₂ (mg/100 g) | 8.45 | 5.05 | Vit. B₆ (mg/100 g) | 10.50 | 15.44 |
| Vit. C (mg/100 g) | 4.91 | 7.13 | Vit. E (mg/100 g, | 23.61 | 31.25 |
| | | | α-TE) | | |
| Vit. K (μg/100 g) | 232.12 | 129.82 | Niacin (mg/100 g, | 5.76 | 2.01 |
| | | | NE) | | |
| Pantothenic | 5.87 | 6.15 | Folic acid | 349.97 | 423.35 |
| acid (mg/100 g) | | | (μg/100 g) | | |

As can be seen from above, the dried ginseng fruit extract and the live ginseng fruit extract have almost the same properties.

### TEST EXAMPLE 3

### Analysis of Lifespan Extending Effect of Ginseng Fruit Extract in Fruit Fly

(1) Breeding of Fruit Fly

Wild-type fruit flies (Oregon-R, Canton-S, Bloomington Drosophila Stock Center (BDSC, Bloomington, Ind.)) were grown in a bottle containing 10% sucrose and 10% yeast (SY) until the imaginal stage. The breeding room was maintained at 25° C. and 50% humidity, with a 12 hr/12 hr light/dark cycle.

(2) Preparation of Sample Containing Medium

SY medium (10% sugar, 10% dry yeast, 0.8% agar, 0.5% antifungal agent, 110 μL/100 mL anti-mite agent) was prepared. Each ginseng fruit extract sample at a concentration of 10 or 25 mg/mL was added to 10% SY medium at 60° C. and the mixture was hardened by cooling. The prepared medium was kept at 4° C. and was used within 2 weeks.

(3) Measurement of Lifespan

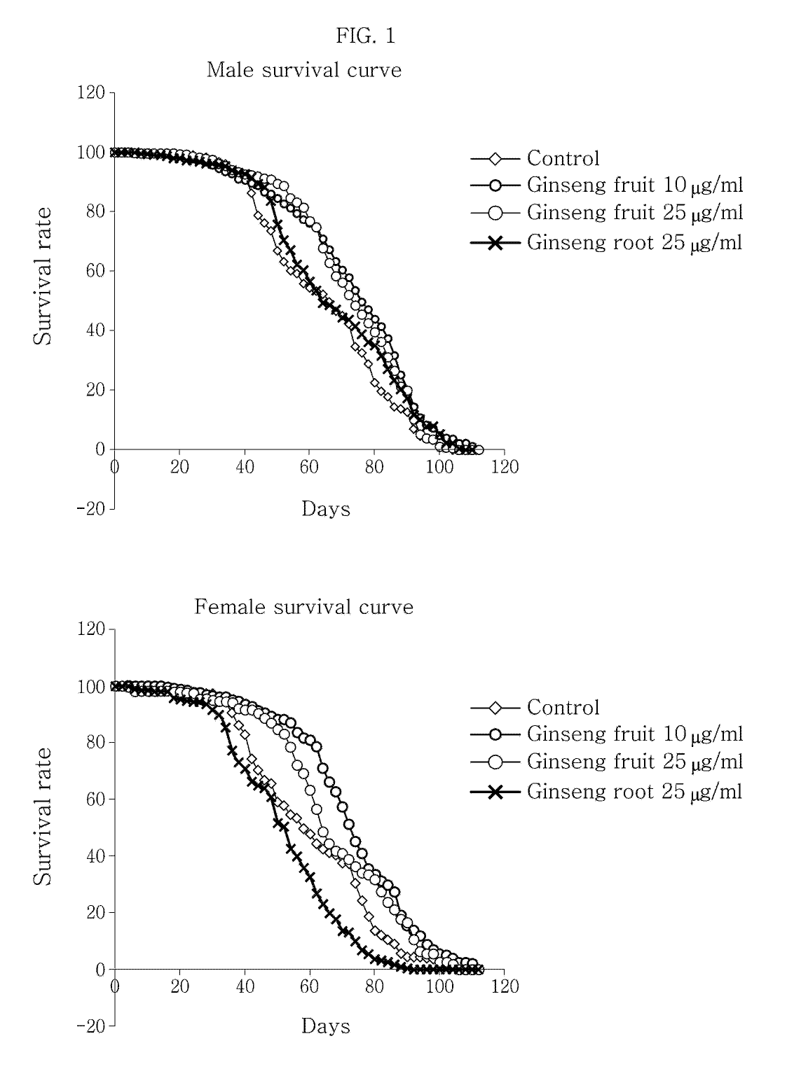

Lifespan was measured after feeding wild-type (Oregon-R, OR) fruit flies with 0 (control) or 10, 25 μg/mL ginseng fruit extract and 25 μg/mL ginseng root extract. Mature female and male (100 each) fruit flies were put in each cage and the number of dead fruit flies was recorded while replacing the medium with fresh one every other day. The experiment was conducted until all the fruit flies in the cage died. The experiment was conducted for each concentration using three cages. The result was statistically analyzed using the Kaplan-Meier (product limit) estimator and the log-rank (generalized Savage, Mantel-Cox) test as shown in FIG. 1.

### TEST EXAMPLE 4

### Analysis of Lifespan Extending Effect of Ginseng Fruit Extract in Nematode

(1) Preparation of Nematode Growth Medium (NGM) Plate

A 955-mL mixture prepared by mixing 3 g of NaCl, 17 g of agar-Difco and 2.5 g of Bacto-peptone was added to an autoclave. After operating the autoclave for 20 minutes, the mixture was cooled to 60° C. while stirring with a magnetic stirrer for 1 hour. Subsequently, 1 mL of 1 M CaCl₂, 1 mL of 3 mg/mL cholesterol in ethanol, 1 mL of 1 M MgSO₄ and 25 mL of 1 M KPO₄ buffer were added and mixed with a magnetic stirrer. The resulting NGM solution was kept at 4° C. on a plate.

(2) Breeding of Caenorhabditis Elegans

Wild-type Caenorhabditis elegans was acquired from the Caenorhabditis Genetic center (University of Minnesota, St. Paul, Minn., USA). Selected eggs were incubated in the NGM plate supplemented with S-basal buffer (100 mM NaCl, 0.01 mM cholesterol, 50 mM potassium phosphate (pH 6.0)) at 20° C. for 16 hours. The hatched eggs (L1 stage larvae) were moved to the NGM plate supplemented with Escherichia coli strain OP50 as food source and were grown until the L4 stage.

(3) Measurement of Lifespan

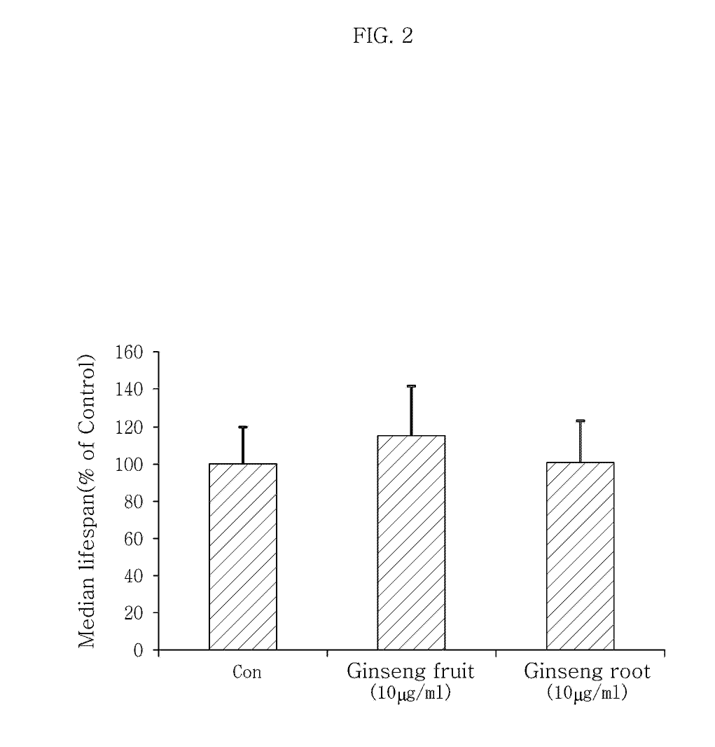

The L4 stage larvae were bred in S-medium [S-basal medium supplemented with 3 mM CaCl₂, 3 mM MgSO₄, 50 mM EDTA, 2 5mM FeSO₄, 10 mM MnCl₂, 10 mM ZnSO₄, 1 mM CuSO₄ and 10 mM KH₂PO₄ (pH 6.0), E. coli OP50] containing 10 mg/mL ginseng fruit extract or 10 mg/mL ginseng root extract. A total of 90 larvae were bred. The number of surviving Caenorhabditis elegans was counted while replacing the medium with fresh one every other day. The result was statistically analyzed using the Kaplan-Meier (product limit) estimator and the log-rank (generalized Savage, Mantel-Cox) test as shown FIG. 2. It can be seen that the ginseng fruit extract is effective in extending the lifespan of the nematode.

### TEST EXAMPLE 5

### Analysis of Lifespan Extending Effect of Ginseng Fruit Extract in Mouse

Because a long time of at least 2 years and an enormous cost are required to measure the actual lifespan of mammals including mouse, various indices have been developed to verify lifespan extending effect.

For examples, the distribution of T-Cell subsets, i.e., the number of CD4 and CD8 memory cells, is used as an aging marker in mammals including human and also is widely used as an index for estimating the lifespan of a subject (Mechanisms of Ageing and Development (2004), 125; 381-390). Specifically, the number of CD4 memory cells increases with aging, and the increase in the number of the cells is associated with faster death regardless of the presence of disease(s) or the cause of the disease(s) (FASEB J. (1997) 11, 775-783).

Accordingly, in the present disclosure, the lifespan extending effect in a mammal was investigated using the distribution of T-Cell subsets.

8-week-old C57BL/6 mice (Central Lab. Animal, Korea) were purchased and stabilized for a week. All the subjects were kept in different cages and were allowed free access to feed and water. The cage was maintained at 22±1° C. and 60±5% humidity, with a 12 hr/12 hr light/dark cycle.

Normal chow diet (D12450B, Research Diets, New Brunswick, N.J., USA) was supplied as the feed, and the amount of feed intake and body weight were measured every week. 50 mg/kg or 100 mg/kg ginseng fruit extract was orally administered to each mouse (10 per group) every day for 6 months. For a control group, only the normal diet was given for 6 months.

6 months later, 400 L of blood was taken from the tail vein of each mouse. After adding 1.5 volume equivalents of PBS, followed by centrifugation at 3000 rpm, only the supernatant was separated. T-cell subset analysis was performed according to the method reported by Miller et al. (J. Gerontol. Biol. Med. Sci. 1997; 11, 775-783) using FITC and PE antibodies (PharMingen, San Diego, Calif., USA). The result is shown in Table 3.

TABLE 3

| | | Ginseng fruit extract | Ginseng fruit extract |
|---|---|---|---|
| Subset | Control | 50 mg/kg/day | 100 mg/kg/day |
| CD4M | 38.6 ± 11.4 | 33.4 ± 5.4 | 30.1 ± 6.8 |
| CD8M | 55.6 ± 15.1 | 50.4 ± 9.2 | 47.3 ± 4.8 |

As can be seen from Table 3, the administration of the ginseng fruit extract resulted in concentration-dependent decrease of the number of the CD4 and CD8 memory cells, which are estimators of the lifespan of the mice.

Hereinafter, formulation examples of the composition according to an aspect of the present disclosure will be described. However, they are for illustrative purposes only and the scope of the present disclosure is not limited by the formulation examples.

### FORMULATION EXAMPLE 1

### Health Food

| | | |
|---|---|---|
| Ginseng fruit extract | 1000 | mg |
| Vitamin mixture | | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Mineral mixture | | |
| Ferrous sulfate | 1.75 | mg |
| Zinc oxide | 0.82 | mg |
| Magnesium carbonate | 25.3 | mg |
| Potassium dihydrogen phosphate | 15 | mg |
| Calcium monohydrogen phosphate | 55 | mg |
| Potassium citrate | 90 | mg |
| Calcium carbonate | 100 | mg |
| Magnesium chloride | 24.8 | mg |

The compositional ratios of the vitamin and mineral mixtures are described as a specific example suitable for a health functional food but may be varied otherwise as desired.

### FORMULATION EXAMPLE 2

### Health Drink

| | |
|---|---|
| Ginseng fruit extract | 1000 mg |
| Citric acid | 1000 mg |
| Oligosaccharide | 100 g |
| Taurine | 1 g |
| Purified water | balance |

The above ingredients were mixed and heated at 85° C. for about 1 hour while stirring according to a commonly employed method for preparing a health drink. The resulting solution was filtered and sterilized.

### FORMULATION EXAMPLE 3

### Tablet

100 mg of a ginseng fruit extract, 50 mg of a soybean extract, 100 mg of glucose, 50 mg of a red ginseng extract, 96 mg of starch and 4 mg of magnesium stearate were mixed and granulated by adding 40 mg of 30% ethanol. After drying at 60° C., the granule was prepared into a tablet.

### FORMULATION EXAMPLE 4

### Granule

100 mg of a ginseng fruit extract, 50 mg of a soybean extract, 100 mg of glucose and 600 mg of starch were mixed and granulated by adding 100 mg of 30% ethanol. After drying at 60° C., the granule was filled in a pouch.

### INDUSTRIAL APPLICABILITY

The present disclosure relates to a composition for life extension, specifically to a composition for life extension, which contains a ginseng fruit extract. The composition of the present disclosure, which contains a ginseng fruit extract as an active ingredient, can extend the lifespan of a subject and thus is highly useful medicinally. In particular, since the ginseng fruit extract as the active ingredient of the composition of the present disclosure has been found to be able to reduce the number of specific cells known as aging markers (Mechanisms of Ageing and Development (2004), 125; 381-390) in mouse, the composition of the present disclosure can extend the lifespan of mammals and thus is highly useful medicinally.

1. A method for life extension comprising a step of administering an effective amount of a composition comprising a ginseng fruit extract to extend life to a subject in need thereof.

2. The method for life extension according to claim 1, wherein the composition comprises 0.001-80 wt % of the ginseng fruit extract based on the total weight of the composition.

3. The method for life extension according to claim 1, wherein the ginseng fruit extract has a ratio of a protopanaxadiol (PD)-based ginsenoside to a protopanaxatriol (PT)-based ginsenoside of 0.5-2.5.

4. The method for life extension according to claim 1, wherein the composition is a pharmaceutical composition.

5. The method for life extension according to claim 1, wherein the composition is a health food composition.
